SEQUENCE LISTING (4 sequences)

SEQ ID NO: 1 - DNA, 285 bp, Canis lupus
atgggctctgagctggagacagcgatggagactctcatcaatgtgttccatgcccactcgggcaaggagggaaacaagtacaagctgagcaagaaggagctaaaggagctgctgcagactgagctctccggcttcctggacgcccagaaggatgcggatgctgtggacaaggtgatgaaagagctagatgagaatggagatggggaggtggacttccaggagtatgtggtgctggtggctgccctcacagtggcctgtaacaacttcttctgggaaaacagttga

SEQ ID NO: 2 - DNA, 285 bp, Felis catus
atgggctcagagctggagacggcgatggagactctcatcaacgtgttccacgcccactcgggcaaggagggagacaagtacaagctgagcaagaaggagctaaaagagctgctgcagaccgagctctctggcttcctggacgcccagaaggatgccgacgctgtggacaaggtgatgaaagagctagacgagaatggagatggggaggtggacttccaagagtatgtggtgctggtggctgccctcacagtggcctgtaacaactttttctgggagaacagttga

SEQ ID NO: 3 - DNA, 1479 bp, Canis lupus
atgcaggaagcgccagccgcgctgcccacggagccgggccccagccccgtgcctgccttcctcggcaagctgtgggcgctggtgggcgacccggggaccgaccacctcatccgctggagcccgagcgggaccagtttcctcgtcagcgaccagagccgcttcgccaaggaagtgctgccccagtacttcaagcacagcaacatggcgagcttcgtgcggcagctcaacatgtacggttttcggaaggtggtgagcatcgagcagggcggcctgctcaggccggagcgcgaccacgtcgagttccagcacccgagcttcgtccgcggccgagagcaactcctggagcgcgtgcggcgcaaggtgcccgcgctgcgcagcgacgacggccgctggcgccccgaggacctgggccggctgctgggcgaggtgcaggctttgcggggagtgcaggagatcaccgaggcgcggctgcgggagctcaggcagcagaacgagatcttatggagggaggtggtgactctgcggcagagccacggtcagcagcatcgcgtcattggcaagctgatccagtgcctctttgggccacttcagacagggtccagcggcgcaggagctaagagaaagctgtctctgatgctggatgaggggagctcatgcccaacaccggccaaattcaacacctgtcctttacctggtgccctcttgcaggatccctactttatccagtcgcccctcccagagaccaccttgggcctcagcagctctcataggaccaggggccctatcatctctgacatccatgaagactctccctcccctgatgggaccaggctttctccttccagtggtggcaggagggagaagggcctggcactgctcaaagaagagccggccagcccagggggggaaggcgaggccgggctggccctggccccaaacgagtgtgacttctgcgtgacagccccccccccactgtccgtggctgtggtgcaggccatcctggaagggaaggggaacttcagccccgaggggcccaggaatgcccaacagcctgaaccaaggggtcccagggaggtacctgacagggggactctgggcctggacaggggggcacgaagcccagagaatctgctgcctcccatgctgcttcgggccccccctgaaagtgtggagcctgcagggcccctggatgtgctgggccccagccatcaagggcgagaatggaccctgatggacttggacatggagctgtccctgatgcagcccttgggtccagagaggagtgagactgagctggcggtcaaggggttaaattctccggggccagggaaggactccacacttggggcaccactcctgctcgatgtccaagcggctttgggaggcccagctctcagccttcctggagctttaaccatttacagcacccctgagagccgagccaactacctaggcccaggggccaatccctccccctga

SEQ ID NO: 4 - DNA, 549 bp, Felis catus
atgggcaatgcgcaggagcggccctcagagacgatcgatcgcgagcggaaacgcctagtggagacgctgcaggacgactccgggctgctgctggatgcactgctggcgcgcggcgtgctcaccgggcctgagtatgaggcgttggacgcgctgcctgatgccgagcgcagggtgcgtcgcctgctgctgctggtacaaagcaagggcgaggccgcctgccaggagctgctgcactgcgcccagcgtactacgcgcgcgccagacccggcctgggactggcagcacgtgggcactggctaccgggaacgcagctacgactctccatgccctggccactggacgcctgaggcacctgacttgaggaccgcttgccccgaaacgcccagagcttcagactgcgacgaggctggggtttcagggggctcggaggcagtatccggaaccctcgaggaactcgatccggaagtggaagctgaagtctctgaaggggctgagccagagccagagccagagcccgactttgaggcgggtgatgagtctgaagattcc

(19) United States
(12) Patent Application Publication
(10) Pub. No.: US 2021/0260215 A1
(43) Pub. Date: Aug. 26, 2021

(54) AAV CARDIAC GENE THERAPY FOR CARDIOMYOPATHY

(71) Applicant: University of Florida Research Foundation, Incorporated, Gainesville, FL (US)

(72) Inventors: Hugh Lee Sweeney, Gainesville, FL (US); Margaret Mary Sleeper, Gainesville, FL (US)

(73) Assignee: University of Florida Research Foundation, Incorporated, Gainesville, FL (US)

(21) Appl. No.: 16/972,740

(22) PCT Filed: Jun. 7, 2019
(86) PCT No.: PCT/US2019/036157

§ 371 Date: Dec. 7, 2020

Related U.S. Application Data
(60) Provisional application No. 62/822,015, filed on Mar. 21, 2019.
(60) Provisional application No. 62/682,772, filed on Jun. 8, 2018.

(51) Int. Cl. A61K 48/00; A61K 31/713; A61K 38/17; C12N 15/113; C12N 15/86

(52) U.S. Cl. CPC A61K 48/005; A61K 31/713; A61K 38/1709; C12N 2750/14143; C12N 15/113; C12N 15/86; A61K 48/0075

(57) ABSTRACT

The present invention is related to compositions and methods useful in treating heart conditions. The disclosed compositions and methods are based on an AAV therapy comprising a recombinant AAV vector for delivering two or more transgenes into the heart of a subject, wherein the transgenes comprise an S100 family protein and an apoptotic inhibitor. In some aspects, targeting multiple sources of one or more heart conditions can provide synergistic benefits during treatment.

### CROSS-REFERENCE TO RELATED APPLICATIONS

This application claims the benefit of the filing dates of U.S. Provisional Application No. 62/682,772 filed Jun. 8, 2018 and U.S. Provisional Application No. 62/822,015 filed Mar. 21, 2019, the entire contents of each of which are incorporated by reference.

### BACKGROUND

Dilated cardiomyopathy (DCM) is the second most common cause of heart disease in dogs, and medical management of the secondary signs is the only therapeutic option. The prognosis for affected dogs depends on the stage of disease and the breed. Doberman pinschers exhibit particularly rapid and uniform progression once congestive heart failure (CHF) has occurred, with most living less than 6 months. Dilated cardiomyopathy (DCM) is the most common type of human cardiomyopathy, occurring mostly in adults 20 to 60. It affects the heart's ventricles and atria, the lower and upper chambers of the heart, respectively. Most forms of DCM are acquired forms from a number of causes that include coronary heart disease, heart attack, high blood pressure, diabetes, thyroid disease, viral hepatitis and viral infections that inflame the heart muscle. Alcohol abuse and certain drugs, such as cocaine and amphetamines, as well as at least two drugs used to treat cancer (doxorubicin and daunorubicin), can also lead to DCM. In addition, there are a number of genetic forms of DCM, including, but not limited to the DCM associated with Duchenne and Becker muscular dystrophies. In the case of certain forms of Becker muscular dystrophy, as well as in most cases of Duchenne muscular dystrophy, the cardiomyopathy can ultimately limit the patient's survival.

### SUMMARY

Cardiomyopathy is the second most common cause of heart disease in subjects and medical management of the secondary signs is the only therapeutic option. The prognosis for affected subjects depends on the stage of disease and the breed. Heart function is critically dependent upon calcium-dependent signaling. During heart disease, malfunctioning of calcium channels within cardiac cells promotes calcium cycling abnormalities, further inhibiting heart function. Gene transfer strategies to reduce calcium cycling abnormalities have been shown to ameliorate heart disease in small and large animal models, as well as in human clinical trials.

In humans, dilated cardiomyopathy is the most common type of cardiomyopathy and can stem from a number of acquired as well as genetic conditions. As in dogs and other animal models, while the origins of the disease are rooted in calcium handling dysfunction, the ultimate progression of the disease is driven by mitochondrial dysfunction and/or stretch-induced apoptosis of the cardiomyocytes. While addressing calcium handling alone may be efficacious at early disease stages, addressing the combination of calcium handling, mitochondrial dysfunction, and apoptosis will be necessary to treat all forms of DCM and at all stages of disease progression.

Disclosed herein are gene delivery approaches for treatment of subjects with cardiomyopathy and congestive heart failure. These approaches comprise the expression of S100A1 to address calcium handling and expression of ARC (Apoptosis Repressor with Caspase Recruitment Domain) to block all sources of apoptosis and normalize mitochondrial function. Expression of S100A1 and ARC transgenes through the disclosed self-complementary AAV vector approach, is rapid (i.e. within hours), which is critical in counteracting the effects of end-stage heart failure, and restricted to the heart. Thus, these approaches address all three drivers of DCM onset and progression and thus should be applicable to any form of DCM at any stage of disease progression.

Some aspects of the present disclosure provide recombinant adeno-associated virus (rAAV) vectors for delivering transgenes into the heart of a subject. In some embodiments, such rAAV vectors include at least two transgenes, one encoding an S100 family protein and one encoding an apoptotic inhibitor. Such rAAV vectors may include, from 5′ to 3′, in order, a first adeno-associated virus (AAV) inverted terminal repeat (ITR) sequence, a promoter operably linked to the transgenes, and a second AAV inverted terminal repeat (ITR) sequence. In some embodiments, two transgenes are operably linked to the same single promoter. In other embodiments, each transgene is operably linked to a separate promoter. In some embodiments, the rAAV vector also includes at least one polyadenylation signal (e.g., 3′ to two transgenes expressed from a single promoter, or 3′ to one or both transgenes expressed from different promoters). Aspects of the disclosure provide recombinant adeno-associated virus (rAAV) nucleic acid vector for delivering two or more transgenes into the heart of a subject, wherein said vector comprises, from 5′ to 3′, in order, a first adeno-associated virus (AAV) inverted terminal repeat (ITR) sequence, two or more transgenes and a promoter operably linked to the two or more transgenes, a polyadenylation signal, and a second AAV inverted terminal repeat (ITR) sequence, wherein the two or more transgenes comprise an S100 family protein and an apoptotic inhibitor.

The transgenes of the present disclosure may be an S100 family protein and an apoptotic inhibitor. For example, the S100 family protein is cardiac S100 calcium-binding protein Al (cS100A1) or a variant thereof. In another example, the apoptotic inhibitor is cardiac Apoptosis Repressor with Caspase Recruitment Domain (cARC) or a variant thereof.

In some embodiments, one or more of the transgenes of the present disclosure are naturally-occurring sequences. In some embodiments, one or more transgenes are engineered to be species-specific. In some embodiments, one or more transgenes are codon-optimized for expression in a species of interest, e.g. canine. In certain embodiments, one or more transgenese (e.g. the cARC transgene) are codon-optimized.

In some embodiments, an Internal Ribosome Entry Site (IRES) is present between the two or more transgenes (e.g., between the cS100A1 transgene and cARC transgene). In some embodiments, the transgene encoding the S100 family protein is 5′ to the transgene encoding the apoptotic inhibitor. In other embodiments, the transgene encoding the apoptotic inhibitor is 5′ to the transgene encoding the S100 family protein.

In some embodiments, the promoter is a cardiac-restricted promoter. The cardiac-restricted promoter may be a promoter from one of the following genes: α-myosin heavy chain gene, 6-myosin heavy chain gene, myosin light chain 2v gene, myosin light chain 2a gene, CARP gene, cardiac α-actin gene, cardiac m2 muscarinic acetylcholine gene, ANF, cardiac troponin C, cardiac troponin I, cardiac troponin T (cTnT), cardiac sarcoplasmic reticulum Ca-ATPase gene, skeletal α-actin; or an artificial cardiac promoter derived from MLC-2v gene. In some embodiments, the cardiac-restricted promoter is a cTnT promoter.

Further provided herein are rAAV particles containing the rAAV vectors disclosed herein, encapsidated in AAV capsids. In some embodiments, the AAV capsid comprises capsid proteins derived from AAV1, AAV2, AAV3, AAV6, AAV8, or AAV9 serotypes. In some embodiments, the AAV capsid comprises capsid proteins derived from the AAVrh.10 serotype.

Other aspects of the present invention include compositions containing the rAAV particles described herein. Such compositions may be administered to a subject for gene therapy for heart disease. In some embodiments, the heart disease causes heart failure in the subject. In some embodiments, the heart disease is cardiomyopathy. In other embodiments, the heart disease is hypertrophic cardiomyopathy or dilated cardiomyopathy. In other embodiments, the heart disease is acute ischemia.

The compositions of the present invention may be administered to the subject via different routes. In some embodiments, the composition is administered via injection into the heart of the subject. In some embodiments, the administration of the composition results in expression of the transgenes in the subject's heart.

In some embodiments, the subject is a mammal. In some embodiments, the mammal is a human. In some embodiments, the mammal is a companion animal. For example, the companion animal may be a dog, cat, horse, pig, cow, sheep, rabbit or other pet.

Each of the elements of the invention may encompass various embodiments of the invention. It is therefore anticipated that each of the limitations of the invention involving any one element, or combinations of elements, may be included in each aspect of the invention. This invention is not limited in its application to the details of construction and the arrangement of components set forth in the following description or as illustrated in the drawings. The present invention is capable of other embodiments and of being practiced or of being carried out in various ways.

### BRIEF DESCRIPTION OF THE DRAWINGS

The following drawings form part of the present specification and are included to further demonstrate certain aspects of the present invention. The disclosure may be better understood by reference to the following description taken in conjunction with the accompanying drawings, in which like reference numerals identify like elements:

FIG. 1 depicts a diagram of an exemplary AAV construct. A first AAV inverted terminal repeat (ITR) is followed by the cardiac troponin T promoter (cTnT), then the codon-optimized sequence for species-specific S100 calcium-binding protein A1 (cS100A1), followed by an internal ribosomal entry site (IRES), followed by the codon-optimized sequence for species-specific Apoptosis Repressor with Caspase Recruitment Domain (cARC), followed by a polyadenylation (PA) sequence, and a second AAV ITR.

FIG. 2 depicts diastolic MRI imaging from a treated muscular dystrophy dog at baseline and several weeks after gene delivery. The data support stable or slightly improved cardiac remodeling with a mild decrease in the diastolic left ventricular volume.

FIG. 3 depicts systolic MRI imaging from a treated muscular dystrophy dog at baseline and several weeks after gene delivery. The data support stable or slightly improved left ventricular systolic function post treatment, with a mild reduction in systolic volume suggesting improved contractility and an increase in left ventricular cardiac output.

FIG. 4 shows ejection fraction, peak strain, and cardiac output of D2.mdx mice after AAVrh.10-S100A1/ARC treatment. Over a 24 week period, mice injected with therapeutic AAV had better maintained ejection fractions, strain development, and cardiac output as compared to sham injected mice.

FIG. 5 shows S100A1 and ARC expression levels in mice treated with recombinant AAVrh.10-S100A1/ARC vector and control mice. Protein analysis (Western blots) confirmed that both S100A1 and ARC levels were elevated in the treated tissues as compared to controls (sham injected).

FIG. 6 shows cardiomyocytes of control and treated mice under 10× and 20× magnification. Cardiac histology data indicates that the treated mice exhibited less DMD pathology as compared to control hearts.

FIG. 7 shows that the first (of two) dystrophin-deficient dogs (GRMD dogs) Calvin showed improved cardiac function after recombinant AAVrh.10-S100A1/ARC treatment. Both injected dogs exhibited improvements in ejection fraction and other cardiac parameters following treatment, measured by cardiac MRI and confirmed by echo data.

FIG. 8 shows data that the second GRMD dog Sebastian showed improved cardiac function after AAVrh.10-S100A1/ARC treatment.

FIGS. 9A to 9C show that AAV-S100A1/ARC treatment decreased serum creatine kinease (CK) levels and prevented muscle atrophy in GRMD dogs Sebastian and Calvin. MRI measurements of limb muscle mass, as measured by the area of both legs (FIG. 9A), maximum cross-sectional area (CSA) (FIG. 9B), and volume of both legs (FIG. 9C). The results demonstrate that skeletal muscle mass had either increased or remained unchanged following cardiac treatment.

FIG. 10 shows that circulating creatine kinase levels (CK) levels in skeletal muscle of the GRMD subjects were reduced after AAVrh.10-S100A1/ARC injection, indicating a reduction in ongoing muscle damage.

### DETAILED DESCRIPTION

The present invention relates to compositions and methods of cardiac gene therapy for heart diseases, e.g., cardiomyopathy, in a subject. The methods of the present invention relate to the use of recombinant AAV (rAAV) particles for the concurrent delivery and expression of two transgenes. The transgenes of the present invention comprise at least two classes of proteins each having specific function to address different aspects of the heart diseases. One class of transgenes regulates the calcium signaling in cardiomyocytes, e.g., the S100 family proteins. The other class of transgenes comprises apoptosis repressors. In some embodiments, the transgenes may be cardiac S100 calcium-binding protein Al (cS100A1) or a variant thereof, and cardiac Apoptosis Repressor with Caspase Recruitment Domain (cARC) or a variant thereof.

The compositions and methods of the present invention are based on, at least in part, the synergistic effects of two transgenes, e.g., S100A1 and ARC, when they are delivered and expressed concurrently in the heart of the subject. The S100A1 protein improves aspects of calcium handling, including normalization of sarcoplasmic reticular calcium transients leading to normalization of contractile function. The ARC protein blocks apoptosis initiated by mitochondrial and nonmitochondrial mechanisms (such as stretch-induced apoptosis), and improves mitochondrial function. In other words, S100A1 and ARC address two separate components of cardiac failure (calcium handling dysfunction and apoptosis) with synergistic benefits, leading to better long-term therapeutic outcomes. Further, the compositions and methods of the present invention are effective at any disease stage of heart failure.

Further provided herein are methods of making rAAV particles suitable for delivering transgenes, e.g., S100A1 and ARC or a variant thereof, into the heart of the subject. Such rAAV particles may comprise a recombinant AAV genome, comprising nucleic acid molecules encoding the transgenes, wherein said nucleic acid molecules are encapsidated by AAV capsid proteins. In some embodiments, the rAAV particles include recombinant adeno-associated virus (rAAV) nucleic acid vector. The recombinant AAV genome is a single-stranded DNA that may further comprise sequence elements that facilitate the integration of the AAV genome into the host genome and the expression of the transgenes. For example, the recombinant AAV genome may comprise tissue-specific promoters to ensure the expression of the transgenes in target tissues or organs. Such rAAV particles may be used in a composition for the treatment of heart conditions.

Thus, the present disclosure further provides recombinant adeno-associated virus (rAAV) vectors for delivering transgenes into the heart of a subject. In some embodiments, the disclosed rAAV vectors include at least two transgenes, one encoding an S100 family protein and one encoding an apoptotic inhibitor. These rAAV vectors may include, from 5′ to 3′, in order, a first adeno-associated virus (AAV) inverted terminal repeat (ITR) sequence, a promoter operably linked to the transgenes, and a second AAV inverted terminal repeat (ITR) sequence. In some embodiments, two transgenes are operably linked to the same single promoter. In other embodiments, each transgene is operably linked to a separate promoter. In some embodiments, the rAAV vector also includes at least one polyadenylation signal (e.g., 3′ to two transgenes expressed from a single promoter, or 3′ to one or both transgenes expressed from different promoters).

The disclosure further provides recombinant adeno-associated virus (rAAV) nucleic acid vector for delivering two or more transgenes into the heart of a subject, wherein said vector comprises, from 5′ to 3′, in order, a first adeno-associated virus (AAV) inverted terminal repeat (ITR) sequence, two or more transgenes and a promoter operably linked to the two or more transgenes, a polyadenylation signal, and a second AAV inverted terminal repeat (ITR) sequence, wherein the two or more transgenes comprise an S100 family protein and an apoptotic inhibitor.

A “transgene”, as used herein, refers to a gene or genetic material that has been transferred naturally, or by any of a number of genetic engineering techniques from one organism to another. A transgene may be a protein or polypeptide of interest (e.g., S100A1, ARC) or an RNA of interest (e.g., a siRNA or microRNA). In some embodiments, one rAAV vector may comprise the coding sequence for one or more transgenes. For example, one rAAV vector may comprise the coding sequence for 1, 2, 3, 4, 5, 6, 7, 8, 9, or 10 transgenes. In some embodiments, the rAAV vectors of the present disclosure comprise the coding sequence of both S100A1 and ARC or variants thereof. In some embodiments, the rAAV vector further comprises a region encoding a Rep protein. The transgenes of the present disclosure comprise two classes of proteins each having specific function to address different aspects of one or more heart conditions. One class of transgenes may regulate the calcium signaling in cardiomyocytes, e.g., the S100 family proteins. Another class of transgenes may comprise apoptosis repressors.

As used herein, the term “variant” refers to a nucleic acid having characteristics that deviate from what occurs in nature, e.g., a “variant” is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type nucleic acid. For instance, a transgene variant is a nucleic acid comprising one or more substitutions in the nucleotides of a transgene, as compared to the wild type sequence thereof. These substitutions may be silent, i.e. they do not modify the amino acid sequence of any encoded protein (or otherwise result in a variant amino acid sequence). Alternatively, these substitutions may result in modifications to the amino acid sequence of an encoded protein, resulting in an encoded protein having one or more amino acid substitutions (e.g., having 1, 2, 3, 4, 5, 6, 7, 8, 9, 10, 10-15, or 15-20 amino acid substitutions) relative to the wild type protein sequence. These substitutions include chemical modifications as well as truncations. In some embodiments, a protein having one or more amino acid substitutions retains wild type protein function, or retains substantially the same function (e.g., at least 25%, at least 50%, at least 75%, e.g. 50-75%, or 75-100% of the function) as the wild type protein function. This term further embraces functional fragments of a wild type nucleic acid sequence.

In some embodiments, one or more of the disclosed transgenes are naturally-occurring sequences. In some embodiments, one or more transgenes are engineered to be species-specific. In some embodiments, one or more transgenes are codon-optimized for expression in a species of interest, e.g., canine. In certain embodiments, the cARC transgene is codon-optimized.

S100 family proteins that may be used in accordance to the present disclosure include, without limitation, S100A1, S100A2, S100A3, S100A4, S100A5, S100A6, S100A7 (e.g., psoriasin), S100A8 (e.g., calgranulin A), S100A9 (e.g., calgranulin B), S 100A10, S 100A11, S100A12 (e.g., calgranulin C), S100A13, S100A14, S100A15 (e.g., koebnerisin), S100A16, S100B, S100P, and S100Z, or variants thereof.

In some embodiments, the S100 family protein may be S100 calcium-binding protein A1 (S100A1). In some embodiments, the S100A1 is cardiac S100A1 (cS100A1) or a variant thereof. The cS100A1 protein is a regulator of myocardial contractility. cS100A1 protein levels are reduced in right ventricular hypertrophied tissue in a model of pulmonary hypertension. Further, S100A1 is a regulator of the genetic program underlying cardiac hypertrophy, in that S100A1 inhibits alphal adrenergic stimulation of hypertrophic genes, including MYH7, ACTA1 and S100B. In cardiomyocytes, S100A1 regulates the calcium-controlled network of SR, sarcomeric, and mitochondrial function through modulation of RyR2, SERCA2, titin, and mitochondrial F1-ATPase activity. As a result, cardiomyocytes and hearts with increased S100A1 expression show increased systolic and diastolic performance, a result of improved Ca²⁺ transient amplitudes resulting from augmented SR Ca²⁺ load and subsequent systolic Ca²⁺ release together with decreased diastolic SR Ca²⁺ leak and enhanced Ca²⁺ resequestration. Concurrently, S100A1 increases mitochondrial high-energy phosphate production and thus coordinates the energy supply with the increased adenosine 5′-triphosphate (ATP) demand by the enhanced cardiomyocyte Ca²⁺ turnover. Reduced S100A1 expression in cardiomyocytes is associated with reduced contractile function, corroborating the pathophysiological significance of this protein.

In some embodiments, the S100A1 cDNA (transgene) sequence has 100% identity to a naturally-occurring S100A1 sequence. In other embodiments, the S100A1 cDNA sequence has at least about 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to a naturally-occurring S100A1 sequence.

In some embodiments, the S100A1 cDNA sequence is engineered to be species-specific. In particular embodiments, the S100A1 cDNA sequence is codon-optimized for expression in a species of interest. Non-limiting examples of S100A1 cDNA sequences are listed below.

S100A1 (canis lupus familiaris)
(NCBI Reference Sequence: XM_005622816.2)
(SEQ ID NO: 1)
ATGGGCTCTGAGCTGGAGACAGCGATGGAGACTCTCATCAATGTGTTCCA
TGCCCACTCGGGCAAGGAGGGAAACAAGTACAAGCTGAGCAAGAAGGAGC
TAAAGGAGCTGCTGCAGACTGAGCTCTCCGGCTTCCTGGACGCCCAGAAG
GATGCGGATGCTGTGGACAAGGTGATGAAAGAGCTAGATGAGAATGGAGA
TGGGGAGGTGGACTTCCAGGAGTATGTGGTGCTGGTGGCTGCCCTCACAG
TGGCCTGTAACAACTTCTTCTGGGAAAACAGTTGA
S100A1 (felis catus)
(NCBI Reference Sequence: XM_003999773.3)
(SEQ ID NO: 2)
ATGGGCTCAGAGCTGGAGACGGCGATGGAGACTCTCATCAACGTGTTCCA
CGCCCACTCGGGCAAGGAGGGAGACAAGTACAAGCTGAGCAAGAAGGAGC
TAAAAGAGCTGCTGCAGACCGAGCTCTCTGGCTTCCTGGACGCCCAGAAG
GATGCCGACGCTGTGGACAAGGTGATGAAAGAGCTAGACGAGAATGGAGA
TGGGGAGGTGGACTTCCAAGAGTATGTGGTGCTGGTGGCTGCCCTCACAG
TGGCCTGTAACAACTTTTTCTGGGAGAACAGTTGA

Some aspects of the application provide compositions and methods that include the delivery of a transgene encoding an apoptotic inhibitor (e.g., an anti-apoptotic agent). Illustrative examples of apoptotic inhibitors include fink, p35, crmA, Bcl-2, Bcl-XL, Mcl-1, E1B-19K from adenovirus, as well as antagonists of pro-apoptotic agents (e.g., antisense, ribozymes, antibodies, etc.). In some embodiments, the apoptotic inhibitor is Apoptosis Repressor with Caspase Recruitment Domain (ARC). In other embodiments, the apoptotic inhibitor is cardiac ARC or a variant thereof. In some embodiments, it may be desirable to deliver an S100 family protein and the apoptotic inhibitor separately. In certain embodiments, a transgene encoding the S100 family protein is delivered concurrently or sequentially with one or more small molecule apoptotic inhibitors. Exemplary small-molecule apoptotic inhibitors include c-Myc inhibitors, Bax inhibitors, p53 inhibitors, tBid inhibitors, caspase inhibitors, and inhibitors of pro-apoptotic BCL-2 family members. In some embodiments, the apoptosis repressor may be cardiac Apoptosis Repressor with Caspase Recruitment Domain (cARC).

The cARC is an apoptotic regulatory protein expressed almost exclusively in myogenic cells. It contains a caspase recruitment domain (CARD) through which it blocks the activation of some initiator caspases. ARC also blocks caspase-independent events associated with apoptosis. Apoptosis caused by acute ischemia and subsequent ventricular remodeling is implicated as a mediator of heart failure. Although postischemic heart failure may have multiple causes, recent attention has been directed toward understanding the contribution of apoptosis or programmed cell death. Apoptosis is characterized by preservation of mitochondrial and sarcolemmal membranes, nuclear chromatin condensation, and phagocytosis by macrophages or neighboring cells without triggering an inflammatory response. The activation of apoptosis is known to occur through mechanisms involving caspases, a family of cysteine proteases that are synthesized as inactive precursors and proteolytically cleaved into their active form. ARC is able the block the activation of apoptosis by blocking the caspases.

In some embodiments, the cARC cDNA (transgene) sequence has identity to a naturally-occurring cARC sequence. In other embodiments, the cARC cDNA sequence has at least about 70% identity, at least about 80% identity, at least about 90% identity, at least about 95% identity, at least about 96% identity, at least about 97% identity, at least about 98% identity, at least about 99% identity, at least about 99.5% identity, or at least about 99.9% identity to a naturally-occurring cARC sequence.

In some embodiments, the cARC cDNA sequence is engineered to be species-specific. In particular embodiments, the cARC cDNA sequence is codon-optimized for expression in a species of interest. In particular embodiments, the cARC cDNA sequence is codon-optimized for expression in canine cells.

The transgene encoding the 5100 family protein (e.g., a cS100A1) may be positioned 5′ to the transgene encoding the apoptotic inhibitor (e.g., a cARC) within the described rAAV nucleic acid vectors. Alternatively, the transgene encoding the apoptotic inhibitor may be positioned 5′ to the transgene encoding the S100 family protein within the described rAAV nucleic acid vectors.

Non-limiting examples of cARC cDNA sequences are listed below.

ARC (canis lupus familiaris)
(NCBI Reference Sequence: NM_001048121.1)
(SEQ ID NO: 3)
ATGCAGGAAGCGCCAGCCGCGCTGCCCACGGAGCCGGGCCCCAGCCCCGT
GCCTGCCTTCCTCGGCAAGCTGTGGGCGCTGGTGGGCGACCCGGGGACCG
ACCACCTCATCCGCTGGAGCCCGAGCGGGACCAGTTTCCTCGTCAGCGAC
CAGAGCCGCTTCGCCAAGGAAGTGCTGCCCCAGTACTTCAAGCACAGCAA
CATGGCGAGCTTCGTGCGGCAGCTCAACATGTACGGTTTTCGGAAGGTGG
TGAGCATCGAGCAGGGCGGCCTGCTCAGGCCGGAGCGCGACCACGTCGAG
TTCCAGCACCCGAGCTTCGTCCGCGGCCGAGAGCAACTCCTGGAGCGCGT
GCGGCGCAAGGTGCCCGCGCTGCGCAGCGACGACGGCCGCTGGCGCCCCG
AGGACCTGGGCCGGCTGCTGGGCGAGGTGCAGGCTTTGCGGGGAGTGCAG
GAGATCACCGAGGCGCGGCTGCGGGAGCTCAGGCAGCAGAACGAGATCTT
ATGGAGGGAGGTGGTGACTCTGCGGCAGAGCCACGGTCAGCAGCATCGCG
TCATTGGCAAGCTGATCCAGTGCCTCTTTGGGCCACTTCAGACAGGGTCC
AGCGGCGCAGGAGCTAAGAGAAAGCTGTCTCTGATGCTGGATGAGGGGAG
CTCATGCCCAACACCGGCCAAATTCAACACCTGTCCTTTACCTGGTGCCC
TCTTGCAGGATCCCTACTTTATCCAGTCGCCCCTCCCAGAGACCACCTTG
GGCCTCAGCAGCTCTCATAGGACCAGGGGCCCTATCATCTCTGACATCCA
TGAAGACTCTCCCTCCCCTGATGGGACCAGGCTTTCTCCTTCCAGTGGTG
GCAGGAGGGAGAAGGGCCTGGCACTGCTCAAAGAAGAGCCGGCCAGCCCA
GGGGGGGAAGGCGAGGCCGGGCTGGCCCTGGCCCCAAACGAGTGTGACTT
CTGCGTGACAGCCCCCCCCCCACTGTCCGTGGCTGTGGTGCAGGCCATCC
TGGAAGGGAAGGGGAACTTCAGCCCCGAGGGGCCCAGGAATGCCCAACAG
CCTGAACCAAGGGGTCCCAGGGAGGTACCTGACAGGGGGACTCTGGGCCT
GGACAGGGGGGCACGAAGCCCAGAGAATCTGCTGCCTCCCATGCTGCTTC
GGGCCCCCCCTGAAAGTGTGGAGCCTGCAGGGCCCCTGGATGTGCTGGGC
CCCAGCCATCAAGGGCGAGAATGGACCCTGATGGACTTGGACATGGAGCT
GTCCCTGATGCAGCCCTTGGGTCCAGAGAGGAGTGAGACTGAGCTGGCGG
TCAAGGGGTTAAATTCTCCGGGGCCAGGGAAGGACTCCACACTTGGGGCA
CCACTCCTGCTCGATGTCCAAGCGGCTTTGGGAGGCCCAGCTCTCAGCCT
TCCTGGAGCTTTAACCATTTACAGCACCCCTGAGAGCCGAGCCAACTACC
TAGGCCCAGGGGCCAATCCCTCCCCCTGA
ARC (felis catus)
(NCBI Reference Sequence: XM_006941587.2)
(SEQ ID NO: 4)
ATGGGCAATGCGCAGGAGCGGCCCTCAGAGACGATCGATCGCGAGCGGAA
ACGCCTAGTGGAGACGCTGCAGGACGACTCCGGGCTGCTGCTGGATGCAC
TGCTGGCGCGCGGCGTGCTCACCGGGCCTGAGTATGAGGCGTTGGACGCG
CTGCCTGATGCCGAGCGCAGGGTGCGTCGCCTGCTGCTGCTGGTACAAAG
CAAGGGCGAGGCCGCCTGCCAGGAGCTGCTGCACTGCGCCCAGCGTACTA
CGCGCGCGCCAGACCCGGCCTGGGACTGGCAGCACGTGGGCACTGGCTAC
CGGGAACGCAGCTACGACTCTCCATGCCCTGGCCACTGGACGCCTGAGGC
ACCTGACTTGAGGACCGCTTGCCCCGAAACGCCCAGAGCTTCAGACTGCG
ACGAGGCTGGGGTTTCAGGGGGCTCGGAGGCAGTATCCGGAACCCTCGAG
GAACTCGATCCGGAAGTGGAAGCTGAAGTCTCTGAAGGGGCTGAGCCAGA
GCCAGAGCCAGAGCCCGACTTTGAGGCGGGTGATGAGTCTGAAGATTCC

Recombinant AAV (rAAV) Vectors

Some aspects of the present invention relate to recombinant AAV vectors that may be used for gene therapy for heart diseases. As used herein, the term “vector” may refer to a nucleic acid vector (e.g., a plasmid or recombinant viral genome), a wild-type AAV genome, or a virus that comprises a viral genome.

The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle. The cap ORF comprises overlapping genes encoding capsid proteins: VP1, VP2 and VP3, which interact together to form the viral capsid. VP1, VP2 and VP3 are translated from one mRNA transcript, which can be spliced in two different manners. Either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. A mature AAV capsid is composed of VP1, VP2, and VP3 (molecular masses of approximately 87, 73, and 62 kDa respectively) in a ratio of about 1:1:10.

Recombinant AAV (rAAV) particles may comprise a recombinant nucleic acid vector (hereafter referred to as “rAAV vector”), which may comprise at a minimum: (a) one or more heterologous nucleic acid regions comprising a sequence encoding a transgene; and (b) one or more regions comprising sequences that facilitate the integration of the heterologous nucleic acid region (optionally with the one or more nucleic acid regions comprising a sequence that facilitates expression) into the genome of the subject. In some embodiments, the sequences facilitating the integration of the heterologous nucleic acid region (optionally with the one or more nucleic acid regions comprising a sequence that facilitates expression) into the genome of the subject are inverted terminal repeat (ITR) sequences (e.g., wild-type ITR sequences or engineered ITR sequences) flanking the one or more nucleic acid regions (e.g., heterologous nucleic acid regions). The ITR sequences may be derived from any AAV serotype (e.g., 1, 2, 3, 4, 5, 6, 7, 8, 9, or 10) or may be derived from more than one serotype. In some embodiments, the ITR sequences are derived from AAV2 or AAV6 serotypes. In some embodiments, a first serotype provided herein is not an AAV2 or AAV8 serotype. In some embodiments, the ITR sequences of the first serotype are derived from AAV3, AAV5 or AAV6. In some embodiments, the ITR sequences are derived from AAV2, AAV3, AAV5 or AAV6. In some embodiments, the ITR sequences are the same serotype as the capsid (e.g., AAV6 ITR sequences and AAV6 capsid, etc.). In some embodiments, the ITR sequences are derived from AAVrh.10 serotype.

ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Calif.; and Addgene, Cambridge, Mass.; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 November 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference). In some embodiments, the rAAV comprises a pTR-UF-11 plasmid backbone, which is a plasmid that contains AAV2 ITRs. This plasmid is commercially available from the American Type Culture Collection (ATCC MBA-331).

In some embodiments, the rAAV vectors of the present invention comprise both the cS100A1 transgene and the ARC transgene, for their concurrent delivery and expression in a subject. Thus, in some embodiments, the rAAV vector comprises one or more regions comprising a sequence that facilitates expression of the transgene (e.g., the heterologous nucleic acid), e.g., expression control sequences operably linked to the nucleic acid. Numerous such sequences are known in the art. Non-limiting examples of expression control sequences include promoters, insulators, silencers, response elements, introns, enhancers, initiation sites, internal ribosome entry sites (IRES) termination signals, and poly(A) signals. Any combination of such control sequences is contemplated herein (e.g., a promoter and a poly(A) signal). In some embodiments, the rAAV vectors comprise a promoter that is operably linked to the coding sequence of the transgenes and facilitates expression of the transgenes.

A “promoter”, as used herein, refers to a control region of a nucleic acid at which initiation and rate of transcription of the remainder of a nucleic acid sequence are controlled. A promoter drives transcription of the nucleic acid sequence that it regulates, thus, it is typically located at or near the transcriptional start site of a gene. A promoter may have, for example, a length of 100 to 1000 nucleotides. In some embodiments, a promoter is operably linked to a nucleic acid, or a sequence of a nucleic acid (nucleotide sequence). A promoter is considered to be “operably linked” to a sequence of nucleic acid that it regulates when the promoter is in a correct functional location and orientation relative to the sequence such that the promoter regulates (e.g., to control (“drive”) transcriptional initiation and/or expression of) that sequence.

Promoters that may be used in accordance with the present invention may comprise any promoter that can drive the expression of the transgenes in the heart of the subject. In some embodiments, the promoter may be a tissue-specific promoter. A “tissue-specific promoter”, as used herein, refers to promoters that can only function in a specific type of tissue, e.g., the heart. Thus, a “tissue-specific promoter” is not able to drive the expression of the transgenes in other types of tissues. In some embodiments, the promoter that may be used in accordance with the present invention is a cardiac-restricted promoter. For example, the promoter may be, without limitation, a promoter from one of the following genes: a-myosin heavy chain gene, 6-myosin heavy chain gene, myosin light chain 2v gene, myosin light chain 2a gene, CARP gene, cardiac α-actin gene, cardiac m2 muscarinic acetylcholine gene, ANF, cardiac troponin C, cardiac troponin I, cardiac troponin T(cTnT), cardiac sarcoplasmic reticulum Ca-ATPase gene, skeletal α-actin; or an artificial cardiac promoter derived from MLC-2v gene.

In some embodiments of the disclosed rAAV vectors, the two or more transgenes are operably controlled by a single promoter. In other embodiments, each of the two or more transgenes are operably controlled by a distinct promoter.

In some embodiments, the rAAV vectors of the present invention further comprise an Internal Ribosome Entry Site (IRES). An IRES is a nucleotide sequence that allows for translation initiation in the middle of a messenger RNA (mRNA) sequence as part of the greater process of protein synthesis. Usually, in eukaryotes, translation can be initiated only at the 5′ end of the mRNA molecule, since 5′ cap recognition is required for the assembly of the initiation complex. In some embodiments, the IRES is located between the transgenes. In such embodiments, the proteins encoded by different transgenes are translated individually (i.e., versus translated as a fusion protein).

In some embodiments, the rAAV vectors of the present disclosure further comprise a polyadenylation (pA) signal. Eukaryotic mRNAs are typically transcribed as a precursor mRNA. The precursor mRNA is processed to generated the mature mRNA, including a polyadenylation process. The process of polyadenylation begins as the transcription of a gene terminates. The 3′-most segment of the newly-made precursor mRNA is first cleaved off by a set of proteins. These proteins then synthesize the poly(A) tail at the RNA's 3′ end. The cleavage site typically contains the polyadenylation signal, e.g., AAUAAA. The poly(A) tail is important for the nuclear export, translation, and stability of mRNA.

In some embodiments, the rAAV vectors of the present invention comprise at least, in order from 5′ to 3′, a first adeno-associated virus (AAV) inverted terminal repeat (ITR) sequence, a promoter operably linked to a first transgene, an IRES operably linked to a second transgene, a polyadenylation signal, and a second AAV inverted terminal repeat (ITR) sequence.

In some embodiments, the rAAV is circular. In some embodiments, the rAAV vector is linear. In some embodiments, the rAAV vector is single-stranded. In some embodiments, the rAAV vector is double-stranded. In some embodiments, the rAAV vector is a self-complementary rAAV vector. Any rAAV vector described herein may be encapsidated by a viral capsid, such as an AAV6 capsid or any other serotype (e.g., a serotype that is of the same serotype as the ITR sequences).

Recombinant AAV (rAAV) Particles

Further provided herein are rAAV particles or rAAV preparations containing such particles. The rAAV particles comprise a viral capsid and an rAAV vector as described herein, which is encapsidated by the viral capsid. Methods of producing rAAV particles are known in the art and are commercially available (see, e.g., Zolotukhin et al. Production and purification of serotype 1, 2, and 5 recombinant adeno-associated viral vectors. Methods 28 (2002) 158-167; and U.S. Patent Application Publication Numbers US 2007/0015238 and US 2012/0322861, which are incorporated herein by reference; and plasmids and kits available from ATCC and Cell Biolabs, Inc.). For example, a plasmid containing the rAAV vector may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

The rAAV particles or particles within an rAAV preparation disclosed herein, may be of any AAV serotype, including any derivative or pseudotype (e.g., 1, 2, 3, 4, 5, 6, 7, 8, 9, 10, 2/1, 2/5, 2/8, 2/9, 3/1, 3/5, 3/8, or 3/9). As used herein, the serotype of an rAAV an rAAV particle refers to the serotype of the capsid proteins of the recombinant virus. In some embodiments, the rAAV particle is rAAV6 or rAAV9. Non-limiting examples of derivatives and pseudotypes include AAVrh.10, rAAV2/1, rAAV2/5, rAAV2/8, rAAV2/9, AAV2-AAV3 hybrid, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A1, Schaffer D V, Samulski R J.). In some embodiments, the rAAV particle is a pseudotyped rAAV particle, which comprises (a) an rAAV vector comprising ITRs from one serotype (e.g., AAV2, AAV3) and (b) a capsid comprised of capsid proteins derived from another serotype (e.g., AAV1, AAV2, AAV3, AAV4, AAV5, AAV6, AAV7, AAV8, AAV9, or AAV10). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

rAAV Gene Therapy for Heart Diseases

The present invention is also directed to compositions comprising one or more of the disclosed rAAV particles or preparations. In some embodiments, the rAAV preparation comprises an rAAV particle comprising a rAAV vector containing ITRs of a first serotype (e.g., AAV3, AAV5, AAV6, or AAV9) and capsid proteins encapsidating the rAAV vector. In some embodiments, the capsid proteins are of the first serotype (e.g., AAV3, AAV5, AAV6, or AAV9). In some embodiments, the preparation has at least a four-fold higher transduction efficiency (e.g., in a human hepatocellular carcinoma cell line, such as Huh7) compared to a preparation prepared using a rAAV vector containing AAV2 ITRs.

As described herein, such compositions may further comprise a pharmaceutical excipient, buffer, or diluent, and may be formulated for administration to host cell ex vivo or in situ in an animal, and particularly a human being. Such compositions may further optionally comprise a liposome, a lipid, a lipid complex, a microsphere, a microparticle, a nanosphere, or a nanoparticle, or may be otherwise formulated for administration to the cells, tissues, organs, or body of a subject in need thereof. Such compositions may be formulated for use in a variety of therapies, such as for example, in the amelioration, prevention, and/or treatment of conditions such as peptide deficiency, polypeptide deficiency, peptide overexpression, polypeptide overexpression, including for example, conditions which result in diseases or disorders as described herein.

The rAAV vectors, rAAV particles, or the composition comprising the rAAV particles of the present disclosure, may be used for gene therapy for heart diseases in a subject in need thereof. Examples of heart disease that may be treated using the methods and compositions of the present invention include, but are not limited to, cardiomyopathy and acute ischemia. In some embodiments, the heart cardiomyopathy is hypertrophic cardiomyopathy or dilated cardiomyopathy. Heart failure caused by cardiomyopathy or other heart diseases, comprise two components, calcium handling dysfunction and apoptosis. The rAAV vectors, particles, and compositions comprising the rAAV particles may be used for treatment of such heart failures when administered to a subject in need thereof, e.g., via direct injection to the heart. The rAAV vectors, particles, and compositions comprising the rAAV particles drive the concurrent expression of cS100A1 protein and ARC proteins in the cardiomyocytes of the subject. S100A1 will improve aspects of calcium handling, including normalization of sarcoplasmic reticular calcium transients leading to normalization of contractile function. ARC will block apoptosis initiated by mitochondrial and nonmitochondrial mechanisms (such as stretch-induced apoptosis), as well as improve mitochondrial function. Thus, the synergistic benefits of the two proteins expressed by the transgenes of the present disclosure can lead to better long-term therapeutic outcomes by targeting both aspects of cardiomyopathy.

Thus, other aspects of the present invention related to administering to a subject in need thereof, the rAAV particles of the present invention. In some embodiments, the number of rAAV particles administered to a subject may be on the order ranging from about 10⁶ to about 10¹⁴ particles/mL or about 10³ to about 10¹³ particles/mL, or any values in between for either range, such as for example, about 10⁶, 10⁷, 10⁸, 10⁹, 10¹⁰, 10¹¹, 10¹², 10¹³, or 10¹⁴ particles/mL. In some embodiments, the number of rAAV particles administered to a subject may be on the order ranging from about 10⁶ to about 10¹⁴ vector genomes(vgs)/mL or 10³ to 10¹⁵ vgs/mL, or any values in between for either range, such as for example, about 10⁶, 10⁷, 10⁸, 10⁹, 10¹⁰, 10¹¹, 10¹², 10¹³, or 10¹⁴ vgs/mL. The rAAV particles can be administered as a single dose, or divided into two or more administrations as may be required to achieve therapy of the particular disease or disorder being treated. In some embodiments, doses ranging from about 0.0001 mL to about 10 mLs are delivered to a subject.

If desired, rAAV particles and rAAV vectors may be administered in combination with other agents as well, such as, e.g., proteins or polypeptides or various pharmaceutically-active agents, including one or more administrations of therapeutic polypeptides, biologically active fragments, or variants thereof. In fact, there is virtually no limit to other components that may also be included, as long as the additional agents do not cause a significant adverse effect upon contact with the target cells or host tissues. The rAAV particles or preparations may thus be delivered along with various other pharmaceutically acceptable agents as required in the particular instance. Such compositions may be purified from host cells or other biological sources, or alternatively may be chemically synthesized as described herein.

Formulations comprising pharmaceutically-acceptable excipients and/or carrier solutions are well-known to those of skill in the art, as is the development of suitable dosing and treatment regimens for using the particular compositions described herein in a variety of treatment regimens, including e.g., oral, parenteral, intravenous, intranasal, intra-articular, and intramuscular administration and formulation.

Typically, these formulations may contain at least about 0.1% of the therapeutic agent (e.g., rAAV particle or preparation, and/or rAAV vector) or more, although the percentage of the active ingredient(s) may, of course, be varied and may conveniently be between about 1 or 2% and about 70% or 80% or more of the weight or volume of the total formulation. Naturally, the amount of therapeutic agent(s) in each therapeutically-useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art when preparing such pharmaceutical formulations. Additionally a variety of dosages and treatment regimens may be desirable.

In certain circumstances, it will be desirable to deliver the rAAV particles or preparations, and/or rAAV vectors in suitably formulated pharmaceutical compositions disclosed herein; either subcutaneously, intracardially, intraocularly, intravitreally, parenterally, subcutaneously, intravenously, intracerebro-ventricularly, intramuscularly, intrathecally, orally, intraperitoneally, by oral or nasal inhalation, or by direct injection to one or more cells (e.g., cardiomyocytes and/or other heart cells), tissues, or organs. In some embodiments, the rAAV particles or the composition comprising the rAAV particles of the present invention are injected directly into the heart of the subject. Direct injection to the heart may comprise injection into one or more of the myocardial tissues, the cardiac lining, or the skeletal muscle surrounding the heart, e.g., using a needle catheter.

The pharmaceutical formulations of the compositions suitable for injectable use include sterile aqueous solutions or dispersions. In some embodiments, the formulation is sterile and fluid to the extent that easy syringability exists. In some embodiments, the form is stable under the conditions of manufacture and storage, and is preserved against the contaminating action of microorganisms, such as bacteria and fungi. The carrier may be a solvent or dispersion medium containing, for example, water, saline, ethanol, polyol (e.g., glycerol, propylene glycol, and liquid polyethylene glycol, and the like), suitable mixtures thereof, vegetable oils or other pharmaceutically acceptable carriers such as those that are Generally Recognized as Safe (GRAS) by the United States Food and Drug Administration. Proper fluidity may be maintained, for example, by the use of a coating, such as lecithin, by the maintenance of the required particle size in the case of dispersion and by the use of surfactants.

The term “carrier” refers to a diluent, adjuvant, excipient, or vehicle with which the rAAV particle or preparation, and/or rAAV vectors is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions may also be employed as liquid carriers.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, intravitreal, subcutaneous and intraperitoneal administration. In this connection, a sterile aqueous medium that can be employed will be known to those of skill in the art in light of the present disclosure. For example, one dosage may be dissolved in 1 mL of isotonic NaCl solution and either added to 1000 mL of hypodermoclysis fluid or injected at the proposed site of infusion, (see, for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by, e.g., FDA Office of Biologics standards.

Sterile injectable solutions are prepared by incorporating the rAAV particles or preparations, Rep proteins, and/or rAAV vectors, in the required amount in the appropriate solvent with several of the other ingredients enumerated above, as required, followed by filtered sterilization. Generally, dispersions are prepared by incorporating the various sterilized active ingredients into a sterile vehicle that contains the basic dispersion medium and the other ingredients from those enumerated above. In the case of sterile powders for the preparation of sterile injectable solutions, the preferred methods of preparation are vacuum-drying and freeze-drying techniques, which yield a powder of the active ingredient plus any additional desired ingredient from a previously sterile-filtered solution thereof.

The amount of rAAV particle or preparation, and/or rAAV vector compositions and time of administration of such compositions will be within the purview of the skilled artisan having benefit of the present teachings. It is likely, however, that the administration of therapeutically-effective amounts of the compositions of the present invention may be achieved by a single administration, such as for example, a single injection of sufficient numbers of infectious particles to provide therapeutic benefit to the patient undergoing such treatment. Alternatively, in some circumstances, it may be desirable to provide multiple or successive administrations of the rAAV particle or preparation, and/or rAAV vector compositions, either over a relatively short, or a relatively prolonged period of time, as may be determined by the medical practitioner overseeing the administration of such compositions.

The compositions of the present invention may include rAAV particles or preparations, and/or rAAV vectors, either alone or in combination with one or more additional active ingredients, which may be obtained from natural or recombinant sources or chemically synthesized. In some embodiments, rAAV particles or preparations are administered in combination, either in the same composition or administered as part of the same treatment regimen, with a proteasome inhibitor, such as Bortezomib, or hydroxyurea.

To “treat” a disease as the term is used herein, means to reduce the frequency or severity of at least one sign or symptom of a disease or disorder experienced by a subject. The compositions described above are typically administered to a subject in an effective amount, which is an amount capable of producing a desired result. The desired result will depend upon the active agent being administered. For example, an effective amount of a rAAV particle may be an amount of the particle that is capable of transferring a heterologous nucleic acid to a host organ, tissue, or cell.

Toxicity and efficacy of the compositions utilized in methods of the present invention may be determined by standard pharmaceutical procedures, using either cells in culture or experimental animals to determine the LD₅₀ (the dose lethal to 50% of the population). The dose ratio between toxicity and efficacy the therapeutic index and it may be expressed as the ratio LD₅₀/ED₅₀. Those compositions that exhibit large therapeutic indices are preferred. While compositions that exhibit toxic side effects may be used, care should be taken to design a delivery system that minimizes the potential damage of such side effects. The dosage of compositions as described herein lies generally within a range that includes an ED50 with little or no toxicity. The dosage may vary within this range depending upon the dosage form employed and the route of administration utilized.

Other aspects of the present invention relate to methods and preparations for use with a subject, such as human or non-human subjects, a host cell in situ in a subject, or a host cell derived from a subject. In some embodiments, the subject is a mammal. In some embodiments, the subject is a companion animal. “A companion animal”, as used herein, refers to pets and other domestic animals. Non-limiting examples of companion animals include dogs and cats; livestock such as horses, cattle, pigs, sheep, goats, and chickens; and other animals such as mice, rats, guinea pigs, and hamsters. In some embodiments, the subject is a human subject. In some embodiments, the subject has or is suspected of having a heart disease that may be treated with gene therapy. In some embodiments, the subject is in any stages of heart failure. In some embodiments, the heart failure is caused by cardiomyopathy. In some embodiments, the heart failure is caused by hypertrophic cardiomyopathy or dilated cardiomyopathy.

The following examples are intended to be illustrative of certain embodiments of the present invention and are intended to be non-limiting. The entire contents of all of the references (including literature references, issued patents, published patent applications, and co pending patent applications) cited throughout this application are hereby expressly incorporated by reference.

### EXAMPLES

### Example 1: Therapeutically Targeting Multiple Aspects of Heart Failure

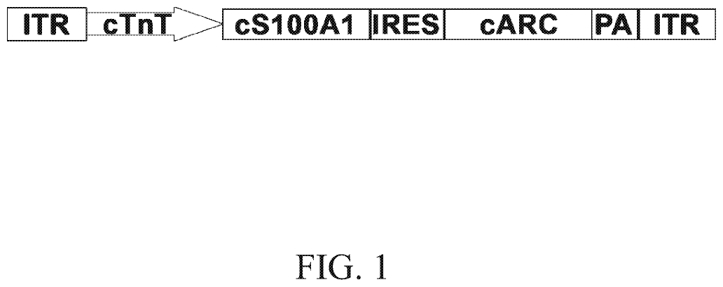

In some aspects, the present invention provides compositions and methods that are useful in treating one or more heart conditions (e.g., cardiomyopathy, hypertrophic cardiomyopathy, dilated cardiomyopathy, heart failure, heart disease, etc.). In some embodiments, compositions provided by the application can be provided to a subject via multiple direct injections into the heart. An exemplary AAV construct that could be provided to a subject is depicted in FIG. 1. In certain embodiments, such an exemplary construct is encapsidated by a recombinant AAV (e.g., AAV6) and comprises species-specific coding sequences of 5100 calcium-binding protein A1 (S100A1) and Apoptosis Repressor with Caspase Recruitment Domain (ARC) to address two separate aspects of one or more heart conditions (e.g., cardiomyopathy). Both transgenes of the exemplary construct in FIG. 1 are driven by the cardiac TnT promoter and thus will only express in cardiomyocytes.

S100A1 will improve aspects of calcium handling, including normalization of sarcoplasmic reticular calcium transients leading to normalization of contractile function. ARC will block apoptosis initiated by mitochondrial and non-mitochondrial mechanisms (e.g., stretch-induced apoptosis), as well as improve mitochondrial function. These two separate components of cardiac failure (calcium handling dysfunction and apoptosis) are addressed separately, but never together. As such, the synergistic benefits of such an approach provide therapeutic options that may result in improved long-term outcomes. By targeting both aspects of cardiomyopathy, compositions and methods provided by the present application may be used to address multiple heart conditions (e.g., hypertrophic or dilated cardiomyopathy), and will be beneficial at any stage of heart failure.

All publications, patents and sequence database entries mentioned in the specification herein are hereby incorporated by reference in their entirety as if each individual publication or patent was specifically and individually indicated to be incorporated by reference. In case of conflict, the present application, including any definitions herein, will control.

### Example 2: Gene Therapy of Dilated Cardiomyopathy in Dogs

Dilated cardiomyopathy (DCM) is the second most common cause of acquired heart disease in dogs, most commonly affecting large breed dogs such as Doberman pinschers, great Danes and Irish Wolfhounds. In humans affected with this disease, there are surgical options such as cardiac transplantation and left ventricular assist devices. However in veterinary medicine the only therapeutic option is medical management of signs associated with heart failure. The prognosis for an affected dog depends on the stage of disease and the breed. For example, most Doberman pinschers live less than 6 months after the development of congestive heart failure (CHF). In contrast other breeds such as cocker spaniels tend to survive longer. As heart disease progresses, malfunctioning of channels that regulate calcium movement within cardiac cells promotes calcium cycling abnormalities, further dysregulating contraction and relaxation of the heart. Notably, calcium transport abnormalities have been recognized in dogs with naturally occurring DCM5 and also occur with heart failure secondary to many different etiologies.

Gene transfer strategies designed to normalize calcium cycling abnormalities ameliorate heart disease in small and large animal models with various forms of heart disease. In fact, clinical trials are already underway in humans to test this therapeutic approach to cardiomyopathy and preliminary results are encouraging. A pilot study is evaluating the efficacy of gene delivery designed to normalize calcium handling in Doberman pinschers affected with DCM and exhibiting CHF. Doberman pinschers are utilized because DCM is widespread in this breed and the disease tends to progress quickly and uniformly in this breed once CHF has developed. Novel modalities to address DCM will have significant impact on all canine breeds predisposed to this idiopathic disease including Doberman pinschers, boxers, great Danes, German shepherds, golden retrievers, etc. Notably, previous investigations into myocardial protein levels in samples from dogs with the most common forms of naturally occurring heart disease (canine degenerative valve disease and DCM) found multiple protein (including S100A1) levels were abnormal (S100A1 was decreased).

These findings suggest that gene delivery targeting S100A1 may effectively treat DCM as well myocardial failure developing secondary to degenerative valve disease. Additionally, apoptosis (programmed cell death) is more common in diseased myocardium and ARC is a potent and multifunctional inhibitor of apoptosis. Currently, the standard of care for veterinary heart failure is the medical management of fluid overload and congestion. Gene delivery techniques directed at abnormal myocardial regulatory molecules offer a mechanistic target that may allow the veterinary clinician to specifically address the myocardial disease process for the first time. Moreover, the cost associated with current vector production techniques and intramyocardial gene delivery of vector make the cost of this therapy within reach for many owners with costs expected to decrease over time.

A minimally invasive method of gene transfer using AAV 2/6 vectors resulted in transduction of >75% of myocardial cells in normal dogs (see Bish L T, Sleeper M M, Brainard B, et al. Percutaneous transendocardial delivery of self-complementary adeno-associated virus 6 achieves global cardiac gene transfer in canines. Mol. Ther. 16, 1953-9 (2008)). Six normal mongrel dogs were treated with either an AAV2 or an AAV6 vector encoding a dominant negative form of Phospholamban (dn-PLN) (a pseudophosphorylated form that competes with the native phospholamban therefore reducing its inhibitory effect on SERCA2a) (n=4) or AAV2/6 do-PLN and S100A1 (n=2). All dogs remained healthy with normal cardiovascular function over 2 years post treatment, indicating that therapy did not cause myocarditis or significantly alter cardiac function, thus supporting the safety of this therapeutic approach. Indeed, over 40 normal and diseased dogs (see below) have been injected, and results to date indicate that the injection technique is well-tolerated. In addition, 20 random canine cases at the Matthew J. Ryan Veterinary Hospital of the University of Pennsylvania were sampled for antibodies to rAAV2/6 and found titers were within the acceptable range for treatment in 19 of the 20 dogs, indicating that prior immune responses will not exclude a significant proportion of therapeutic candidates. To determine if this therapeutic approach was efficacious for treatment of DCM, Portuguese water dogs with a severe form of rapidly progressing juvenile DCM were then treated. Notably, dogs injected with AAV2/6 dn-PLN exhibited a marked decrease in phosphorylated PLN, supporting the potential ability of this approach to normalize calcium cycling in this disease model. Moreover, gene delivery with a vector containing both do-PLN and S100A1 slowed the development of CHF secondary to DCM to a greater degree than did delivery of a vector containing do-PLN alone. The combination vector delayed onset of CHF by an average of 4 weeks as compared to do-PLN therapy alone. For this reason, the combined vector approach is utilized in a pilot study to determine if gene therapy is effective in prolonging the life of Dobermans affected with adult-onset DCM and congestive heart failure.

The study has a blinded, placebo controlled design. Based on the last 12 Doberman pinscher cases of DCM and CHF that have been treated, there was a mean survival of 148 days (standard deviation of 160 days). Using a power of 0.8, alpha (2 sided) of 0.05 and a ratio of cases to controls of 1, a sample size of 13 dogs in each group are required to detect a difference in 6 month survival. This calculation was determined using a parametric sample size test. Twenty six Doberman pinschers with DCM and controlled CHF are enrolled. In order to be eligible for enrollment, the dog must have a circulating neutralizing antibody titer to AAV2/6 of less than 1:20 and be clear of extra-cardiac disease. Additionally, dogs with concomitant congenital heart disease or evidence of primary mitral valvular disease are excluded. At baseline (time of enrollment) an antibody titer, CBC, and chemistry panel is used for screening purposes. Dogs undergo a 3-minute electrocardiogram (ECG) and a complete echocardiogram (ECHO) and owners complete a previously validated quality of life questionnaire. The ECG is evaluated for interval duration and the presence of arrhythmias. The ECHO includes 2D, M-mode and Doppler studies (including tissue Doppler). Thoracic radiographs are used to stage the disease (dogs are clinically compensated with a history of congestive heart failure).

Dogs fulfilling the requirements for enrollment are randomly assigned to the placebo arm (cardiac injection with saline) or the gene therapy group (cardiac injection with AAV2/6-ARC-s100a1). Standard medical management for DCM and congestive heart failure continues throughout the study in all dogs (pimobendan, angiotensin inhibitor and diuretic therapy). Saline instead of empty capsid is used as the sham therapy so that control dogs can undergo gene delivery if the treatment group demonstrates a significant improvement compared to the placebo group. At 2, 4, 6, 9, and 12 months following therapy ECG, ECHO, a quality of life questionnaire and laboratory analyses are repeated. Statistical analysis is performed at bi-monthly intervals.

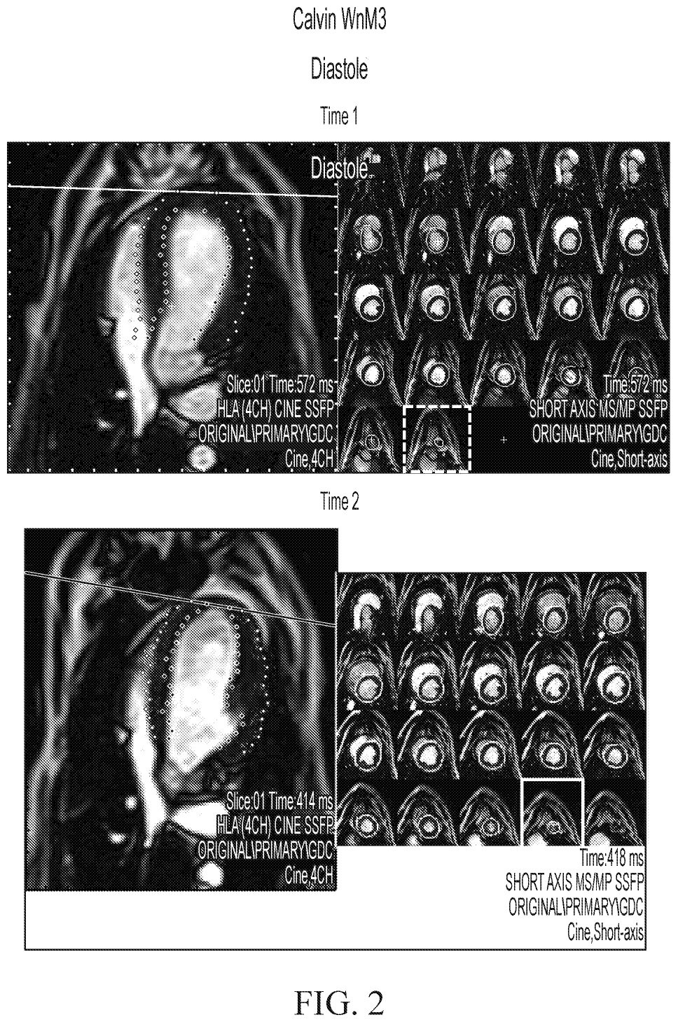

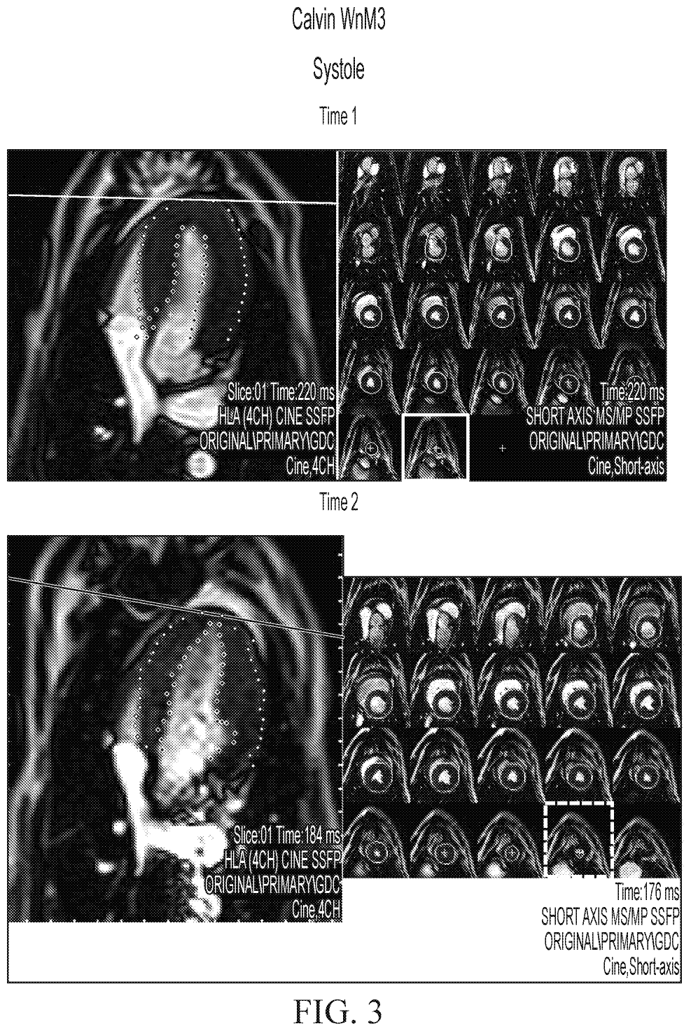

FIGS. 2 and 3 depict diastole (relaxation) and systole (contraction) data, respectively, in a treated muscular dystrophy dog. The endocardial and epicardial contours can be seen in each of the figures. The data indicates stable or slightly improved function post treatment over several weeks as seen in Table 1. Table 1, below, shows the left ventricular mass (LVM [g]), end diastolic volume (EDV [ml]), end systolic volume (ESV [ml]), stroke volume (SV [ml]), ejection fraction (EF [%]), and cardiac output (CO [1/min]) results for the data taken at times 1 (pre-treatment) and time 2 (post-treatment).

TABLE 1

| Acquisition Date | LVM[g] | EDV[ml] | ESV[ml] | SV[ml] | EF[%] | CO[l/min] |
|---|---|---|---|---|---|---|
| Time 2 | 91.395035 | 54.22289 | 24.595001 | 29.627889 | 54.640926 | 3.940509 |
| Time 1 | 87.251524 | 57.471229 | 25.660014 | 31.811215 | 55.351548 | 3.117499 |

### Example 3: Assessment of Dystrophy Phenotypes Following Vector Delivery Into Mice and Dogs

Cardiac AAV gene delivery of the S100A1/ARC self-complementary vector was assessed in mouse and dog models of Duchenne muscular dystrophy (dystrophin-deficiency). Earlier, the AAV8 (including multiple variants thereof), AAV9, and AAVrh.10 serotypes were compared in their ability to infect canine hearts, and AAVrh.10 was found to be the most efficient. For this reason, AAVrh.10 was used for all experiments described in this Example.

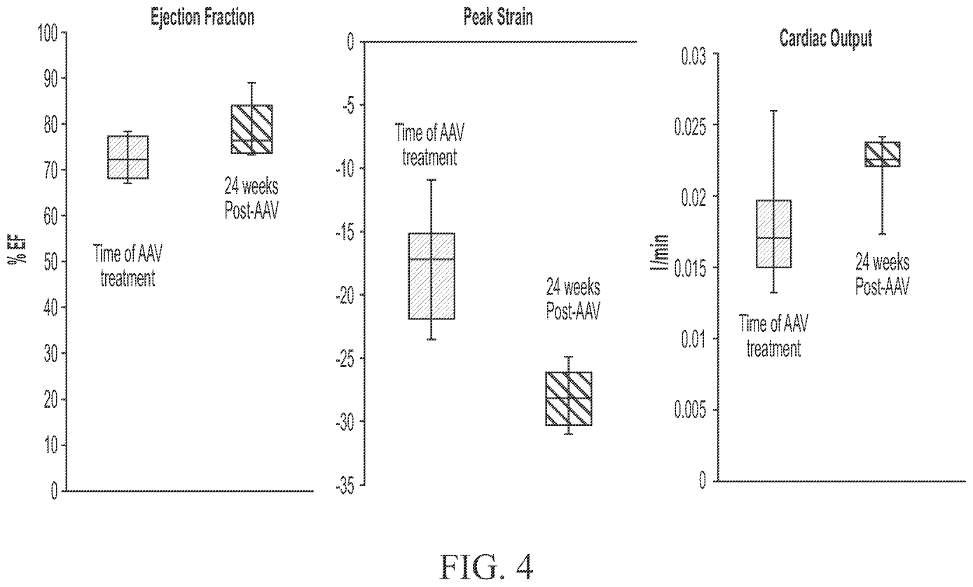

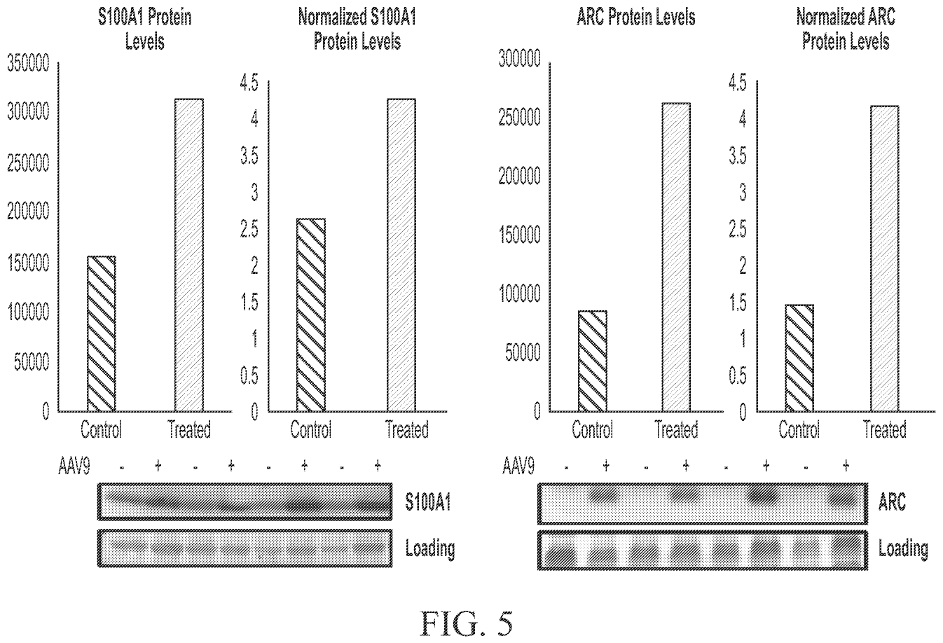

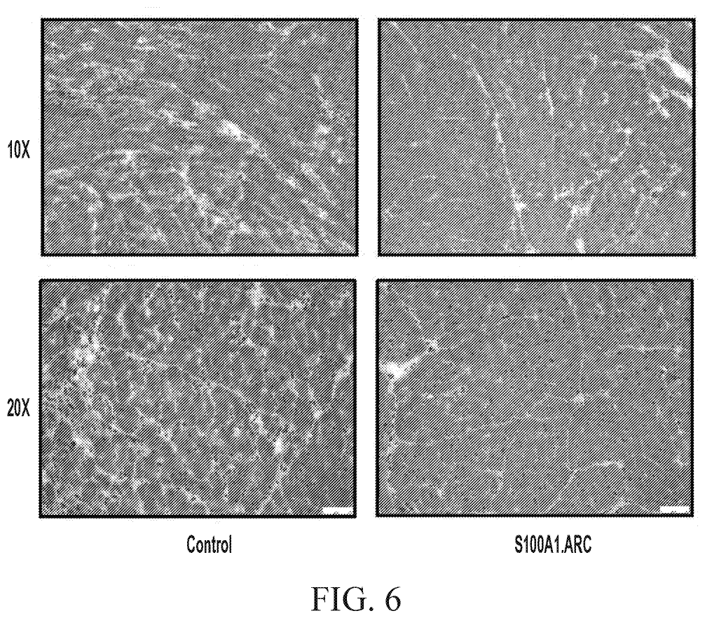

Mdx (dystrophin-deficient) mice on the DBA/2J background (“D2.mdx”) were injected at 4 weeks of age with recombinant AAVrh.10-S100A1/ARC vector (referred to below as the “therapeutic AAV”) and sacrificed 24 weeks later. D2.mdx mice recapitulate several human characteristics of Duchenne muscular dystrophy myopathy, such as reduced lower hind limb muscle mass, atrophied myofibers, increased fibrosis and inflammation, and muscle weakness. Over this 24 week period, the mice injected with the therapeutic AAV had better maintained ejection fractions, strain development, and cardiac output as compared to sham injected mice (see FIG. 4), as measured by cardiac MRI. Protein analysis (Western blots) confirmed that both S100A1 and ARC levels were elevated in the treated tissues as compared to controls (sham injected) (see FIG. 5). Furthermore, cardiac histology demonstrated that the treated hearts demonstrated much less pathology as compared to control hearts (see FIG. 6).

Two GRMD (dystrophin-deficient) dogs were injected with the therapeutic vector at the time of first decrease in their cardiac ejection fractions—a symptom indicating onset of cardiomyopathy. Earlier findings from a natural history study of dog subjects indicated that, as soon as ejection fractions begin to fall, they continue to fall progressively over the next year. Dogs typically do not survive longer than 8-12 months after the ejection fraction begins this steady decrease.

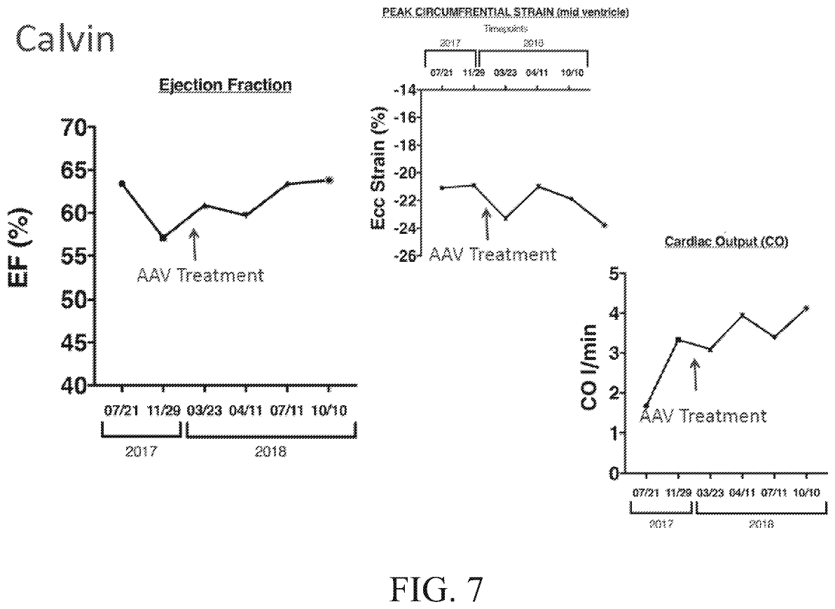

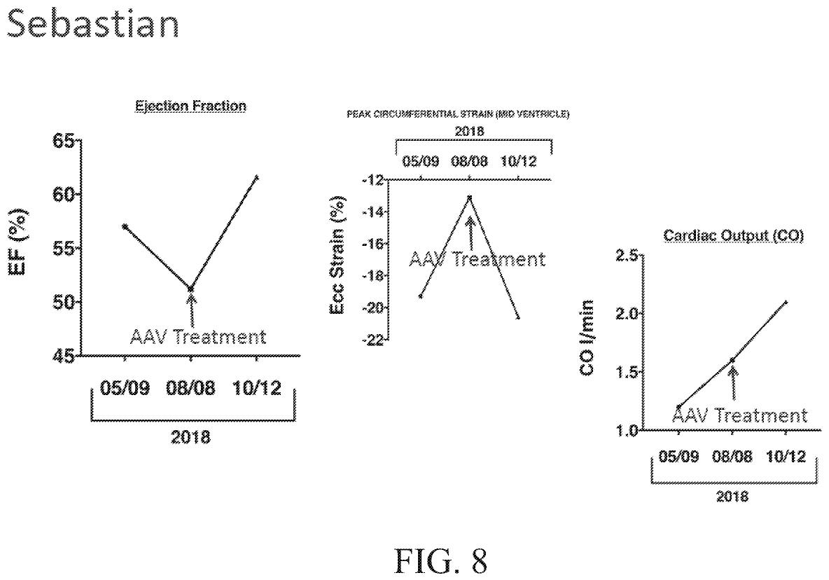

As shown in FIGS. 7 and 8, both subjects showed improvements in ejection fraction and other cardiac parameters several months after treatment with AAVrh.10-S100A1/ARC, as measured by cardiac MRI and confirmed by echo measurements. Nearly 12 months after treatment, the first subject exhibited a steady ejection fraction within the normal range. Likewise, nearly 7 months after treatment, the second subject exhibited a steady, normal ejection fraction.

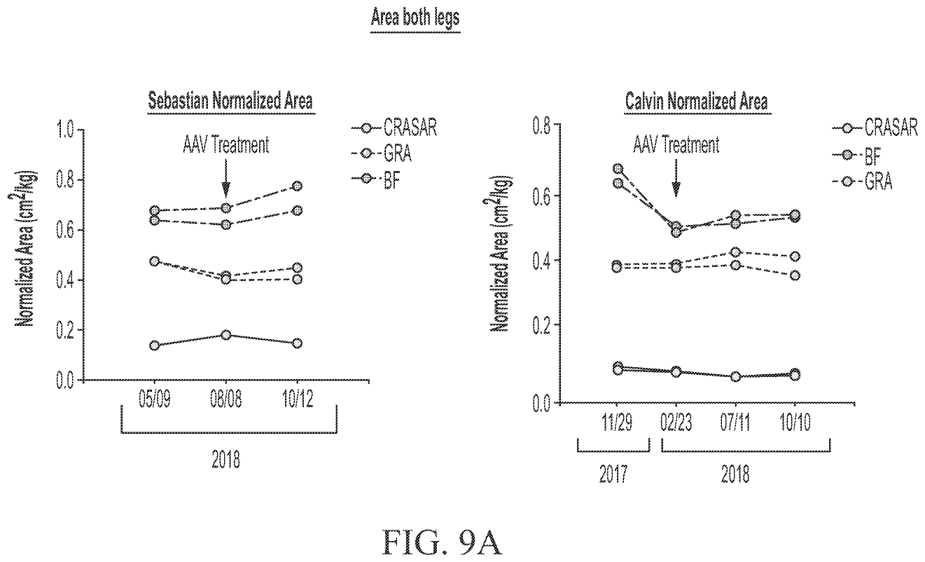

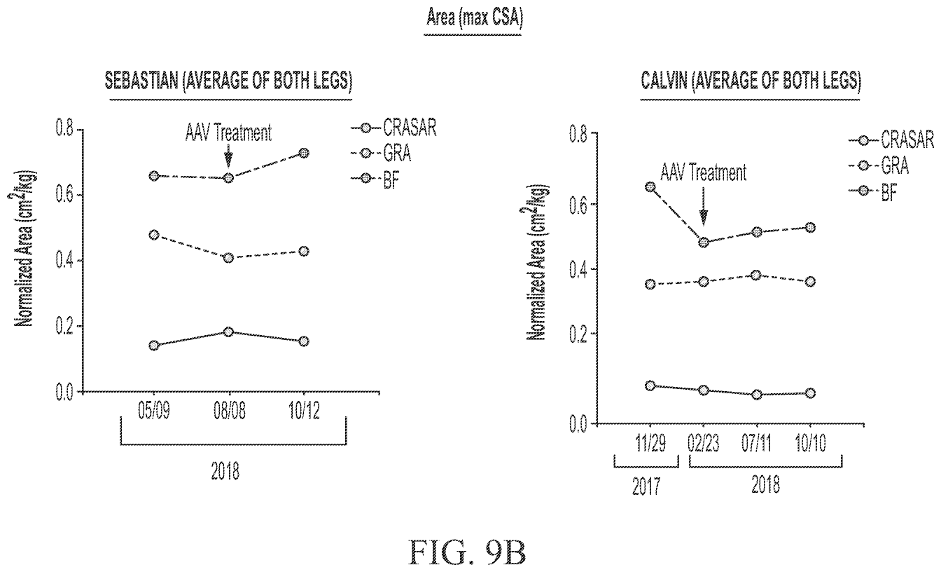

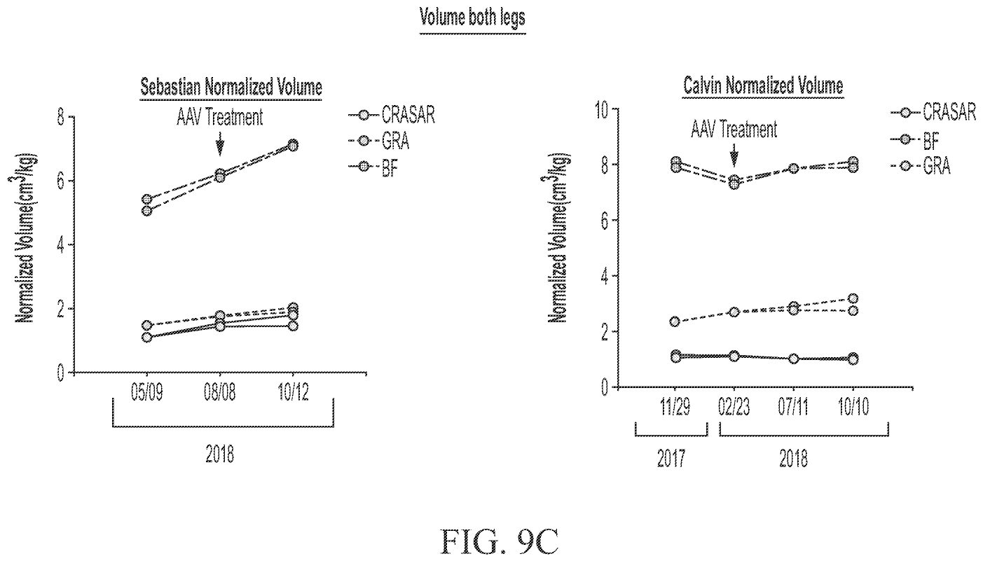

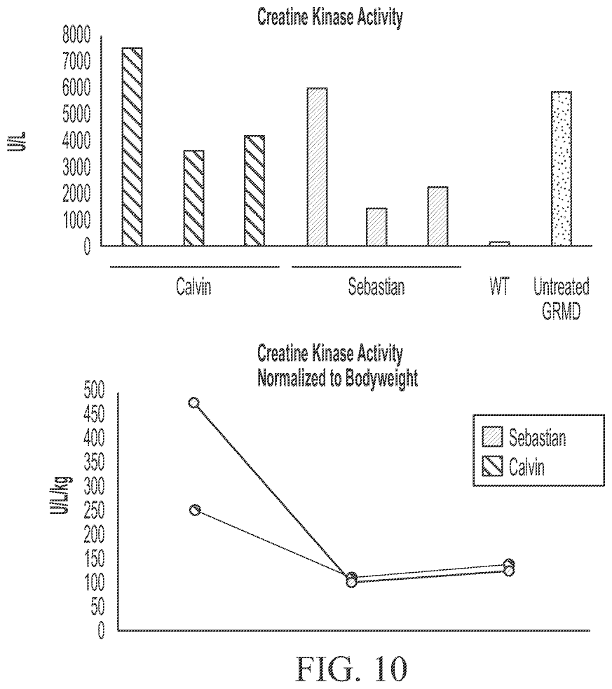

Not only was cardiac function improved, but there was also a constant improvement in the exercise capacity of the dogs, as evaluated qualitatively by filming the subjects during exercise. Consistent with this improved exercise capacity, MRI measurements of the subjects' limbs demonstrated that skeletal muscle mass was either augmented or unchanged following AAV treatment (FIGS. 9A to 9C). In addition, circulating creatine kinase levels (CK) levels in skeletal muscle was reduced post-treatment (FIG. 10), indicating that a reduction in ongoing muscle damage.

### Example 4: Treatment of Additional Dog Subjects

Two Doberman pinscher subjects have been treated with AAVrh.10-S100A1/ARC to date, wherein both dogs had experienced heart failure at the time of treatment. The first dog was near death at the time of treatment, exhibiting a cardiac ejection fraction of only 10%. Within 24 hours post-treatment, the ejection fraction improved to 25% (data not shown). At the dog's first follow up visit at 4 months post-treatment, the ejection fraction had held steady at 26%. This subject was still living 5 months post-treatment.

The second treated Doberman pinscher had an ejection fraction of 32% prior to treatment—a fraction that is low, but not in immediate danger of death. The dog's ejection fraction improved to 49% within 24 hours following treatment (data not shown), which is within normal range. The second dog was reported to be doing well 5 weeks post-treatment. This subject had a first follow up visit at 4 months post-treatment.

Based on these preliminary findings, AAVrh.10-S100A1/ARC treatment is able to restore cardiac function in canines to normal range.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Multiple embodiments of the present invention are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A recombinant adeno-associated virus (rAAV) nucleic acid vector for delivering two or more transgenes into the heart of a subject, wherein said vector comprises, from 5′ to 3′, in order, a first adeno-associated virus (AAV) inverted terminal repeat (ITR) sequence, two or more transgenes and a promoter operably linked to the two or more transgenes, a polyadenylation signal, and a second AAV inverted terminal repeat (ITR) sequence, wherein the two or more transgenes comprise an S100 family protein and an apoptotic inhibitor.

2. The rAAV vector of claim 1, wherein the S100 family protein is cardiac S100 calcium-binding protein A1 (cS100A1) or a variant thereof.

3. The rAAV vector of claim 1 or 2, wherein the apoptotic inhibitor is cardiac Apoptosis Repressor with Caspase Recruitment Domain (cARC) or a variant thereof.

4. The rAAV vector of claim 2 or 3, wherein an Internal Ribosome Entry Site (IRES) is present between the cS100A1 transgene and cARC transgene.

5. The rAAV vector of any one of claims 1-4, wherein the transgenes are species-specific.

6. The rAAV vector of any one of claims 1-5, wherein the promoter is a cardiac-restricted promoter.

7. The rAAV vector of claim 6, wherein the cardiac-restricted promoter is selected from the group of genes consisting of: a-myosin heavy chain gene, 6-myosin heavy chain gene, myosin light chain 2v gene, myosin light chain 2a gene, CARP gene, cardiac a-actin gene, cardiac m2 muscarinic acetylcholine gene, ANF, cardiac troponin C, cardiac troponin I, cardiac troponin T (cTnT), cardiac sarcoplasmic reticulum Ca-ATPase gene, and skeletal a-actin, and an artificial cardiac promoter derived from MLC-2v gene.

8. The rAAV vector of claim 6 or 7, wherein the cardiac-restricted promoter is cTnT.

9. An rAAV particle comprising the rAAV vector of any one of claims 1-8 encapsidated in an AAV capsid.

10. The rAAV particle of claim 9, wherein the AAV capsid comprises capsid proteins derived from AAV1, AAV2, AAV3, AAV6, AAV8, or AAV9 serotypes.

11. The rAAV particle of claim 9, wherein the AAV capsid comprises capsid proteins derived from AAVrh.10 serotype.

12. A composition comprising the rAAV particle of any one of claims 9-11.

13. A method of treatment of a subject suffering from a heart disease comprising administering to the subject the composition of claim 12 or the rAAV particle of any one of claims 9-11.

14. The method of claim 13, wherein the heart disease causes heart failure in the subject.

15. The method of claim 13 or 14, wherein the heart disease is cardiomyopathy.

16. The method of any one of claims 13-15, wherein the heart disease is hypertrophic cardiomyopathy or dilated cardiomyopathy.

17. The method of claim 13 or 14, wherein the heart disease is acute ischemia.

18. The method of any one of claims 13-17, wherein the composition is administered via injection into the heart of the subject.

19. The method of any one of claims 13-18, wherein the administering of the composition results in expression of the two or more transgenes in the subject's heart.

20. The method of any one of claims 13-19, wherein the subject is a mammal.

21. The method of claim 20, wherein the mammal is a human.

22. The method of claim 20, wherein the mammal is a companion animal.

23. The method of claim 22, wherein the companion animal is a dog or a cat.

24. The rAAV vector of any one of claims 1-8, wherein the transgene comprising an S100 family protein is positioned 5′ to the transgene comprising an apoptotic inhibitor.

25. The rAAV vector of any one of claims 1-8, wherein the transgene comprising an apoptotic inhibitor is positioned 5′ to the transgene comprising an S100 family protein.
